(19) United States
(12) Patent Application Publication
(10) Pub. No.: US 2015/0017682 A1
(43) Pub. Date: Jan. 15, 2015

(54) ANASTOMOTIC LEAKAGE DETECTION THROUGH DUAL ACTION CATHETER

(71) Applicant: Empire Technology Development LLC, Wilmington, DE (US)

(72) Inventor: Quentin Arthur Carl Adam, North Parramatta (AU)

(21) Appl. No.: 14/378,959

(22) PCT Filed: Nov. 9, 2012
(86) PCT No.: PCT/US12/64326

§ 371 Date: Aug. 14, 2014

(51) Int. Cl. A61B 5/00; A61B 10/00; A61M 25/00

(52) U.S. Cl. CPC A61B 5/4255; A61B 5/6852; A61M 25/0043; A61B 10/0045; A61M 2205/15; A61B 2010/0061
USPC 435/34; 600/581

(57) ABSTRACT

Technologies are generally described for detecting the presence of anastomotic leakage in the abdominal cavity through a dual action catheter placed in the vicinity of a surgery site. A portion of the dual action catheter may be affixed around the surgery site, for example, coiled around the surgery site on the intestine. Through openings on the catheter near the surgery site, a neutral fluid such as saline solution may be injected and extracted through the same openings resulting in retrieval of fluids in the same are. By testing the extracted fluids, chemical and biochemical activity within the abdominal cavity may be determined in order to gauge the start of any anastomotic leakage.

### BACKGROUND

Unless otherwise indicated herein, the materials described in this section are not prior art to the claims in this application and are not admitted to be prior art by inclusion in this section.

Surgeries performed on internal organs such as the stomach, the intestines, or even the esophagus may involve removal of parts of the organ and reattachment of other parts. For example, the location of the sutures and/or staple line, where remaining ends of the colon are joined together following surgery is referred to as the anastomosis. Anastomotic leakage is a common complication after colorectal or similar surgeries, and may occur at the location of the anastomosis, during which gases and/or fluids may leak from inside of the organ into the abdominal cavity potentially causing severe illness and even death. The anastomotic leakage may occur post-operatively as the organs resume their operations and internal pressure increases putting stress on the anastomosis allowing gas and/or fluids to leak out at the anastomosis location. Often times, the initial leakage may go undetected until severe symptoms including peritonitis, abscess, sepsis, and even death occur.

While early detection of anastomotic leakage after surgery may enable early intervention and help avoid severe complications, illness, and death due to the leakage, a majority of current approaches to managing anastomotic leakage include monitoring clinical symptoms such as fever, breathing and neurologic irregularities, and even sepsis. However, these symptoms may occur weeks after surgery, and long after the initial leakage has transpired.

### SUMMARY

The present disclosure generally describes techniques for detecting the presence of leakage in the abdominal cavity through a dual action catheter placed in a vicinity of a surgery site. According to some embodiments, the present disclosure describes a method for detecting presence of anastomotic leakage inside a body. An example method may include positioning a dual action catheter in a vicinity of a surgery site, causing a neutral fluid to be injected into abdominal cavity near the surgery site through openings on the dual action catheter, causing abdominal cavity fluids near the openings on the dual action catheter to be extracted, and testing the extracted abdominal cavity fluids for presence of one or more substances indicating the anastomotic leakage.

According to other embodiments, the present disclosure also describes a system for detecting presence of anastomotic leakage inside a body. An example system may include a dual action catheter positioned in a vicinity of a surgery site in an abdominal cavity and a dual action syringe system. The dual action catheter may release a neutral fluid into the abdominal cavity near the surgery site through openings on the dual action catheter and collect abdominal cavity fluids near the openings on the dual action catheter. The dual action syringe system may inject the neutral fluid into the dual action catheter and extract the abdominal cavity fluids from the dual action catheter such that the extracted abdominal cavity fluids are tested for presence of one or more substances indicating the anastomotic leakage.

According to further embodiments, the present disclosure describes a dual action catheter for detecting presence of anastomotic leakage inside a body. The dual action catheter may include a first portion with at least one lumen, where the first portion is arranged to couple to a dual action syringe system outside a body for receiving a neutral fluid to be injected into an abdominal cavity in a vicinity of a surgery site and providing abdominal cavity fluids extracted from the vicinity of the surgery site. The dual action catheter may also include a second portion with at least one lumen, where the second portion is arranged to couple to the first portion and to release the neutral fluid into the abdominal cavity in the vicinity of the surgery site and collect abdominal cavity fluids through openings on the second portion such that the extracted abdominal cavity fluids are tested for presence of one or more substances indicating the anastomotic leakage.

According to yet other embodiments, the present disclosure describes a computer readable memory device with instructions stored thereon for controlling a dual action catheter based system to detect presence of anastomotic leakage inside a body. The instructions may include causing a neutral fluid to be injected into abdominal cavity near a surgery site through openings on a dual action catheter, where the catheter is positioned in a vicinity of the surgery site; causing abdominal cavity fluids near the openings on the dual action catheter to be extracted; and testing the extracted abdominal cavity fluids for presence of one or more substances indicating the anastomotic leakage.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates an example anastomotic leakage detection system setup, where a dual action catheter may be placed inside the body and syringe activator placed outside the body for dual action detection of leakage at surgery sites;

FIG. 2 illustrates an example anastomotic leakage detection setup, where a dual action catheter and syringe system may be controlled by a controller and extracted fluids tested for leakage symptoms;

FIG. 3 illustrates an example dual lumen, punctured helical catheter that may be placed around a surgery site of the intestines to detect anastomotic leakage;

FIG. 4 illustrates two additional example catheter configurations that may be placed at a surgery site of the intestines to detect anastomotic leakage;

FIG. 5 illustrates details of an example dual lumen, punctured catheter that may be placed around a surgery site of the intestines to detect anastomotic leakage and two example cross-sectional views of the tube;

FIG. 6 illustrates an example dual lumen, punctured tape-style catheter that may be secured around a surgery site with adhesive wings;

FIG. 7 illustrates another example dual lumen, punctured catheter that may have a substantially flat shape when deployed;

FIG. 8 illustrates two example cross-sectional views of a catheter according to FIG. 6 and FIG. 7;

FIG. 9 illustrates an example dual action syringe that may be used a catheter according to FIG. 3 through FIG. 7;

FIG. 10 illustrates a general purpose computing device, which may be used to control a leakage detection system implementing a dual action catheter inside the body;

FIG. 11 illustrates a special purpose controller, which may be used to control a dual action syringe and leakage sensors;

FIG. 12 is a flow diagram illustrating an example method that may be performed by a computing device such as the computing device in FIG. 7; and

FIG. 13 illustrates a block diagram of an example computer program product, all arranged in accordance with at least some embodiments described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to methods, apparatus, systems, devices, and/or computer program products related to detecting the presence of leakage in the abdominal cavity through a dual action catheter placed in a vicinity of a surgery site.

Briefly stated, technologies are generally described for detecting the presence of anastomotic leakage in the abdominal cavity through a dual action catheter placed in the vicinity of a surgery site. A portion of the dual action catheter may be affixed around the surgery site, for example, coiled around the surgery site on the intestine. Through openings on the catheter near the surgery site, a neutral fluid such as saline solution may be injected and extracted through the same openings resulting in retrieval of fluids in the same are. By testing the extracted fluids, chemical and biochemical activity within the abdominal cavity may be determined in order to gauge the start of any anastomotic leakage.

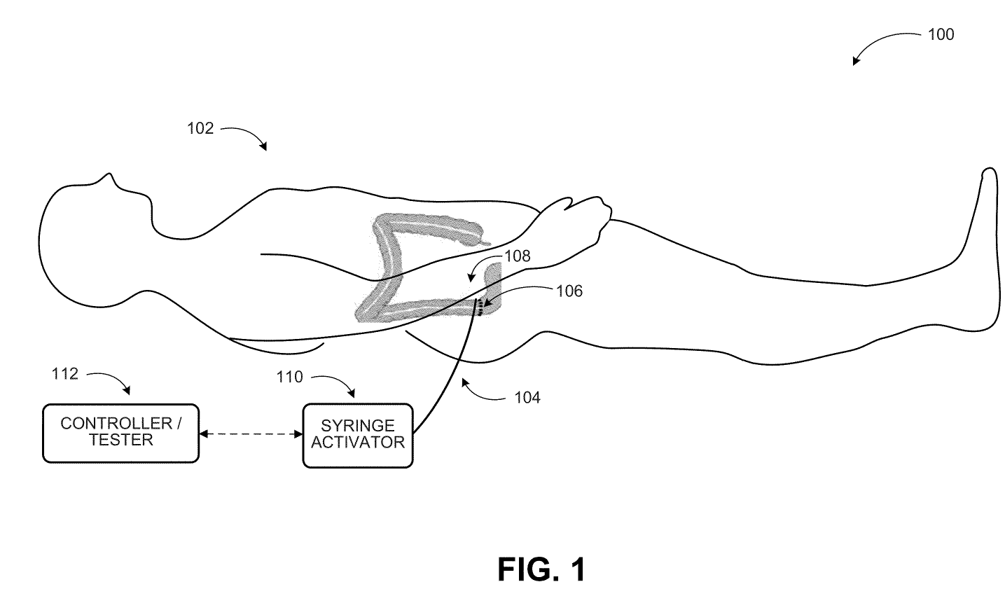

FIG. 1 illustrates an example anastomotic leakage detection system setup, where a dual action catheter may be placed inside the body and syringe activator placed outside the body for dual action detection of leakage at surgery sites, arranged in accordance with at least some embodiments described herein.

Diagram 100 illustrates an example anastomotic leakage detection system setup, where a dual action catheter 104 may be placed inside the body 102 around the area of surgery (106) on the large intestine, for example, and a syringe activator device 110 placed outside the body 102 coupled to a controller/tester 112. Anastomotic leakage is a common complication after colorectal or similar surgeries, during which a portion of an internal organ may be removed and reattached. During some surgeries, a portion of an internal organ such as the stomach, the small intestine, the large intestine, or even the esophagus may be removed or otherwise operated on and then reattached together (or anastomosed). The location of the sutures and/or staple line where the reattachment occurs is referred to as the anastomosis. After the surgery, anastomotic leakage may occur at the location of the anastomosis, during which gases and/or fluids may leak from inside of the organ into the abdominal cavity, for example, the intraperitoneal space potentially causing severe illness and even death. The anastomotic leakage can occur post-operatively as the body resumes normal operation (e.g., gas production) and the internal pressure within the operated organ may increase putting pressure on the anastomosis and allowing gas and/or fluids to leak into the abdominal cavity at the anastomosis location.

Often times, the initial leakage may go undetected until severe symptoms including peritonitis, abscess, sepsis, and even death occur. Early detection of anastomotic leakage after surgery is important for enabling early intervention and management of the anastomosis in order to avoid severe complications, illness, and death due to the leakage. Additionally, it should be noted that the colon and the gastrointestinal tract is not the only sites where an anastomosis can be performed, and it may also be possible to sever and reconnect blood vessels, and to perform similar procedures in the urinary tract, as some examples.

The portion of the dual action catheter 104 placed around the anastomosis may include openings, through which a neutral fluid such as saline solution or purified water may be injected. According to example embodiments, a neutral fluid or solution may be selected with suitable properties such as pH balanced for internal usage, safe for intrabody use, resorbable by the body, etc. For example, medical saline solution is typically a single blend. However, there may be less optimal blends that are also tolerated by the body. Some blends may be less desirable depending on which substances are tested in the collected fluid to detect anastomosis.

Subsequently or simultaneously, fluids in the same area may be extracted through the same openings. The extracted fluids may include a mixture of the injected neutral fluid and any substances that are found just outside the surgery site. References are made herein to the “area” around the surgery site. The term “area” is intended to indicate the three-dimensional space around the surgery site. For example, a surgery site on the colon may include a substantially cylindrical or spherical space around the colon in the vicinity of the surgery site. If there is leakage at the surgery site, injected fluids may move up, down, sideways, etc. in space around the surgery area. Thus, fluids may be extracted in that space. The extracted fluids may be subsequently tested for known substances such as proteins, unprocessed carbohydrates, or acidity increase to determine if there is an indication of leakage from the operated organ into its surroundings. The testing of the chemical and biochemical activity within the abdominal cavity may also include detection of ingested substances such as dyes, radioactive substances, activated charcoal, etc. in order to gauge the start of any anastomotic leakage.

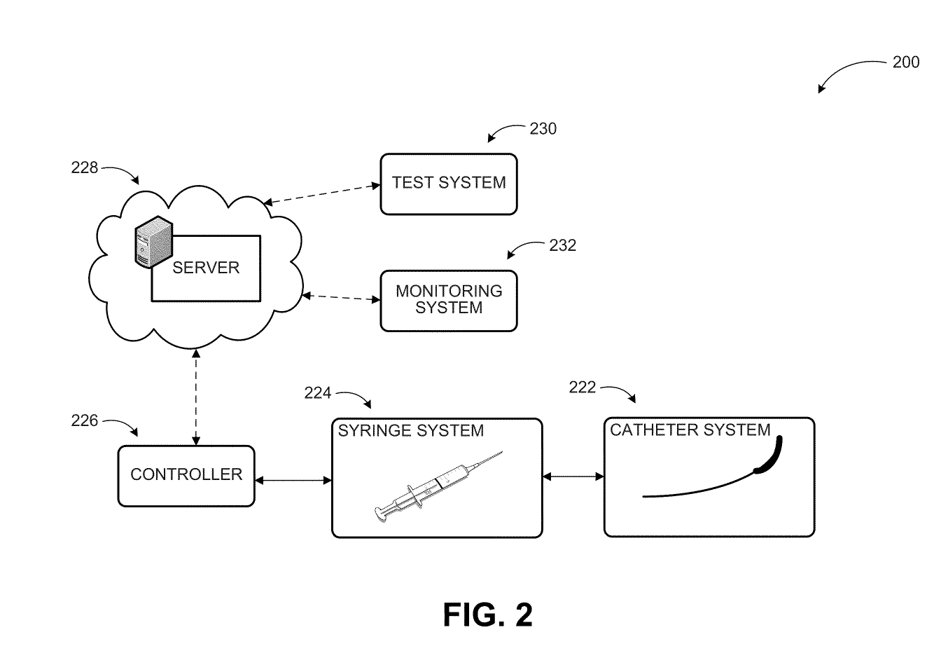

FIG. 2 illustrates an example anastomotic leakage detection setup, where a dual action catheter and syringe system may be controlled by a controller and extracted fluids tested for leakage symptoms, arranged in accordance with at least some embodiments described herein.

As shown in diagram 200, major components of a system according to some embodiments may include a catheter system 222, a syringe system 224, and a controller 226. In some embodiments, a basic configuration may include a dual action catheter that can inject the neutral fluid and extract the abdominal cavity fluids and a manually operated syringe mechanism, for example, a double cylinder syringe to perform the injection and the extraction simultaneously through manual action. In other embodiments, the syringe(s) may be controlled through the controller 226, which may communicate with a monitoring system 232 and/or a testing system 230 via one or more networks 228 (managed by one or more servers). In some example systems, the controller 226 may be coupled with a test device that may promptly test the extracted fluids and provide results to the monitoring system 232. The controller 226 may also receive instructions from the monitoring system 232 and/or test system 230, transmit alerts if leakage is detected, perform calibration measurements, compute statistical analyses, and so on.

The catheter system 222 may include a single or dual lumen catheter, a portion of which may be affixed around the surgery site through sutures, staples, or similar techniques. The portion placed at the surgery site may be helically wound around the anastomosis in a few loops or allowed to be placed at the anastomosis in a meandering form. Alternatively, the end portion of the catheter may include two or more branches that may be affixed along the surgery site (e.g., longitudinally with the intestine) such that as much of the surgery site (e.g., the circumference of the intestine) is covered. The end portion of the catheter placed around the surgery site may include a number of openings such as puncture holes to release the neutral fluid and extract the abdominal cavity fluids.

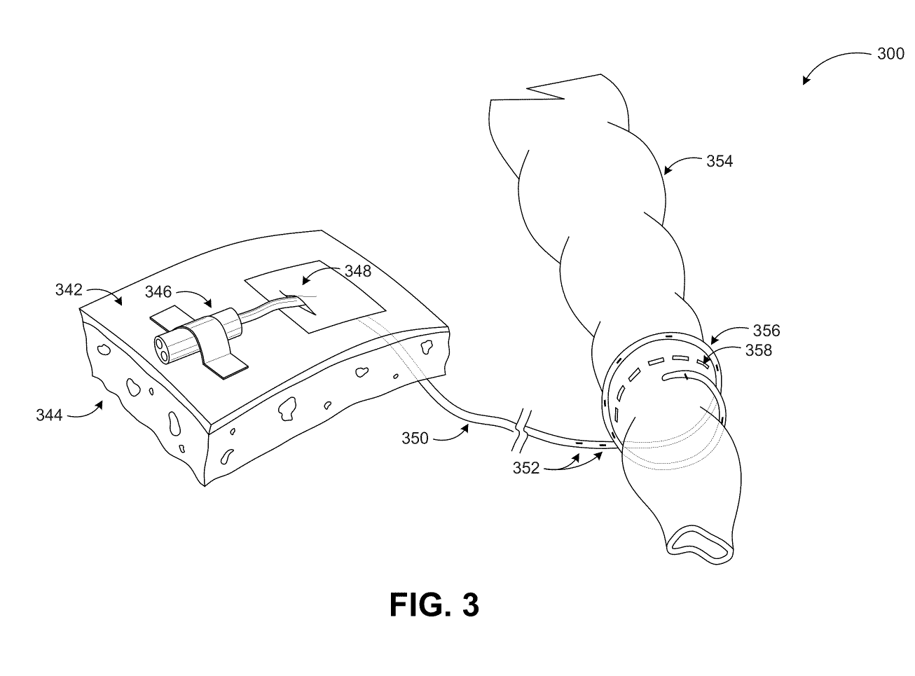

FIG. 3 illustrates an example dual lumen, punctured helical catheter that may be placed around a surgery site of the intestines to detect anastomotic leakage, arranged in accordance with at least some embodiments described herein.

Diagram 300 depicts an example dual action catheter 350, which may be inserted into the body through an incision 348 in the skin 342 and underlying tissue 344. One end of the dual action catheter 350 may be coupled to a connector 346, which may be affixed on the skin and provide removable connection to a syringe system for injecting neutral fluid and extracting abdominal cavity fluids. For example, the syringe system may be replaced after each injection/extraction cycle.

In the example configuration illustrated in diagram 300, a portion of the dual action catheter inside the body may be wrapped around the anastomosis 358 on the large intestine 354 in one or two loops 356. The loops 356 may be attached to the large intestine 354 through absorbable sutures such that the dual action catheter can be removed through a gentle tugging (“rip cord”) action.

The dual action catheter 350 may have a number of openings 352 at its portion that is placed around the anastomosis 358 such that the neutral fluid can be released into the area and abdominal cavity fluids can be extracted. The openings 352 may be puncture holes or similar prefabricated openings. A number, a separation, and a size of the openings 352 may be determined based on the type and size of the catheter, type of surgery, the organ that is operated on, etc.

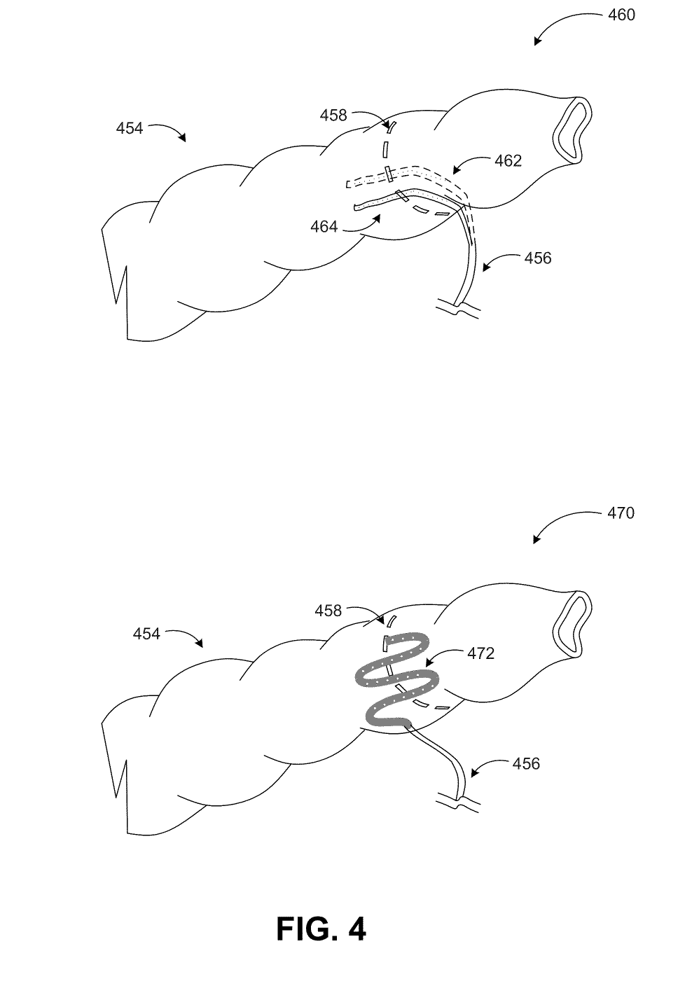

FIG. 4 illustrates two additional example catheter configurations that may be placed at a surgery site of the intestines to detect anastomotic leakage, arranged in accordance with at least some embodiments described herein.

As discussed above, one configuration for placing a dual action catheter around the anastomosis may be forming one or more loops around the operated organ at the anastomosis, where the catheter may be removed through a tugging action once its use is completed and the attachment mechanism (e.g., absorbable sutures) is removed. In some scenarios, there may be a concern about removing the dual action catheter through a tugging action, for example, in surgeries involving the small intestine. Furthermore, the wrap-around style placement may not be practical in other scenarios, for example, in surgeries involving the stomach. In such cases, or whenever the wrap-around configuration is not desired/practical to be used, other placement configurations may be employed.

Diagram 460 in FIG. 4 illustrates an alternative configuration employing multiple branches. In the example configuration, an end portion of the dual action catheter 456 may include two or more branches 462, 464, which may be places at the surgery site 458 with some distance between them (e.g., equidistance). Thus, the branches may cover a larger three-dimensional space compared to a single catheter placed flatly against the surgery site increasing the likelihood of detection of anastomotic leakage. For example, if the leakage were to occur on a small part of an anastomosis in the large intestine 454, a single dual action catheter placed transversally to the anastomosis may have a smaller chance to detect the leakage compared to two, three, or more branches of the catheter that may be placed equidistantly around the large intestine 454. In such a configuration, all branches of the dual action catheter may include similar size and number of openings to ensure consistency and reliability of measurements.

Diagram 470 illustrates yet another alternative configuration, where a single branch dual action catheter 456 may be placed around the anastomosis 458 in a meandering form 472. The meandering form portion of the catheter may be positioned to cover a portion of the circumference of the organ (e.g., the large intestine 454) such that removal of the catheter even by a tugging action is less likely to harm the organ.

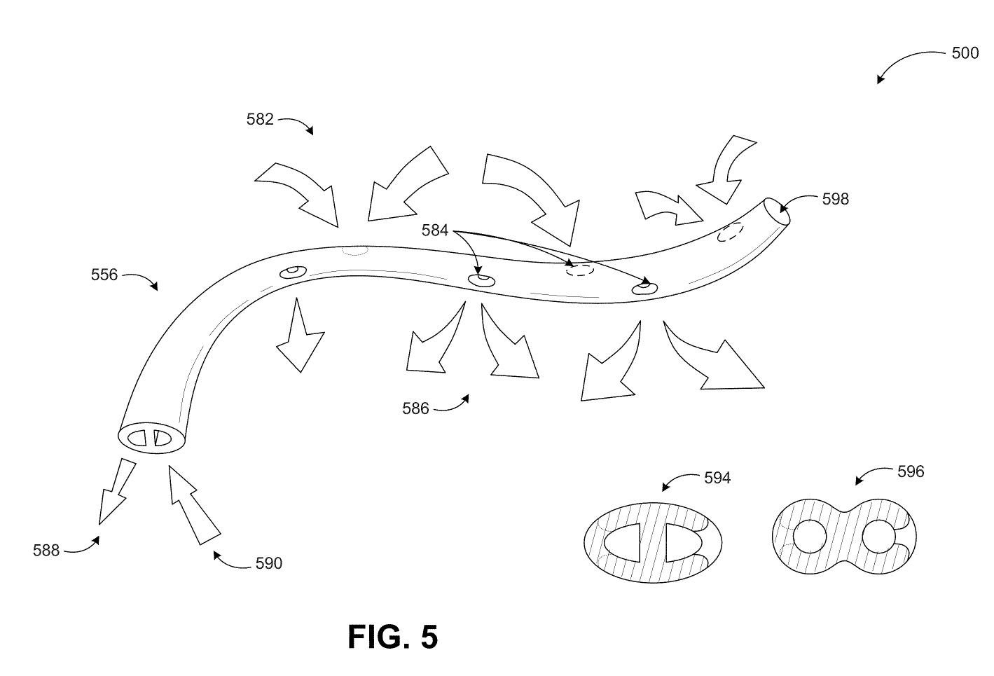

FIG. 5 illustrates details of an example dual lumen, punctured catheter that may be placed around a surgery site of the intestines to detect anastomotic leakage and two example cross-sectional views of the tube, arranged in accordance with at least some embodiments described herein.

Diagram 500 includes an end portion of a dual action catheter 556 with openings 584 for injection 586 of the neutral fluid into the surrounding area and extraction 582 of abdominal cavity fluids from the surrounding area. This may be accomplished by sealing an end 598 of the dual action catheter and injecting the neutral fluid into the catheter, then applying negative pressure to the catheter to extract fluids from the surrounding area.

In a basic example, the dual action catheter may be a simple tube, where the neutral fluid may be injected first and released through the openings 584, followed by a second action of extracting the abdominal cavity fluids through negative pressure. In another example configuration, shown in diagram 500, the dual action catheter may include two lumens 588 and 590. One of the lumens (e.g., 590) may be used to inject the neutral fluid and the other (e.g., 588) may be used to extract the abdominal cavity fluids. In this configuration, the injection and the extraction operations may be performed sequentially or simultaneously. A dual lumen configuration catheter may have a variety of cross-sectional shapes. Two example cross-sections 594 and 596 are illustrated in FIG. 5, but embodiments are not limited to those, and any suitable cross-section shape may be selected for the dual action catheter.

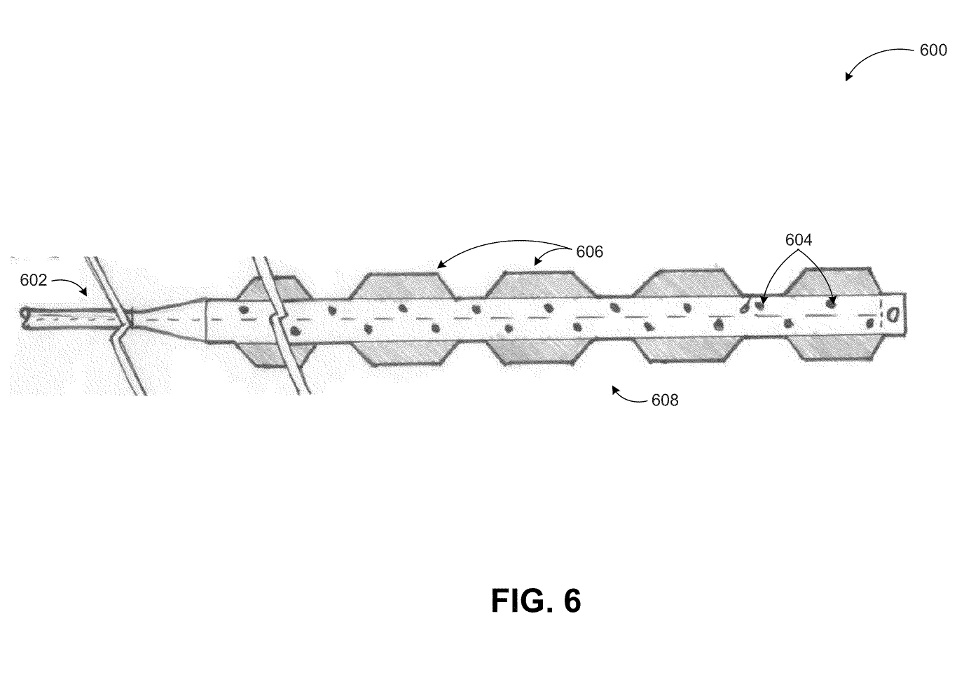

FIG. 6 illustrates an example dual lumen, punctured tape-style catheter that may be secured around a surgery site with adhesive wings, arranged in accordance with at least some embodiments described herein.

Diagram 600 depicts another example dual action catheter, which may include a tubular section 602 and a substantially flat section 608. The flat section 608 may be wrapped around the anastomosis area (e.g., on the colon) allowing the holes 604 to permanently face inwards towards the colon section and to prevent the catheter from rolling. The flat section 608 of the catheter may be secured to the colon (and to itself to form a band) using an adhesive tape in some examples. The adhesive tape may be formed as wings 606 on either side of the flat section 604.

In some example embodiments, the adhesive tape may be absorbable and be absorbed into the body so the catheter can be removed through a gentle tugging action. A syringe system may be coupled to the tubular section 602 of the catheter outside the body for injecting neutral fluid and extracting abdominal cavity fluids. The syringe system may be removable and be replaced after each injection extraction cycle, for example.

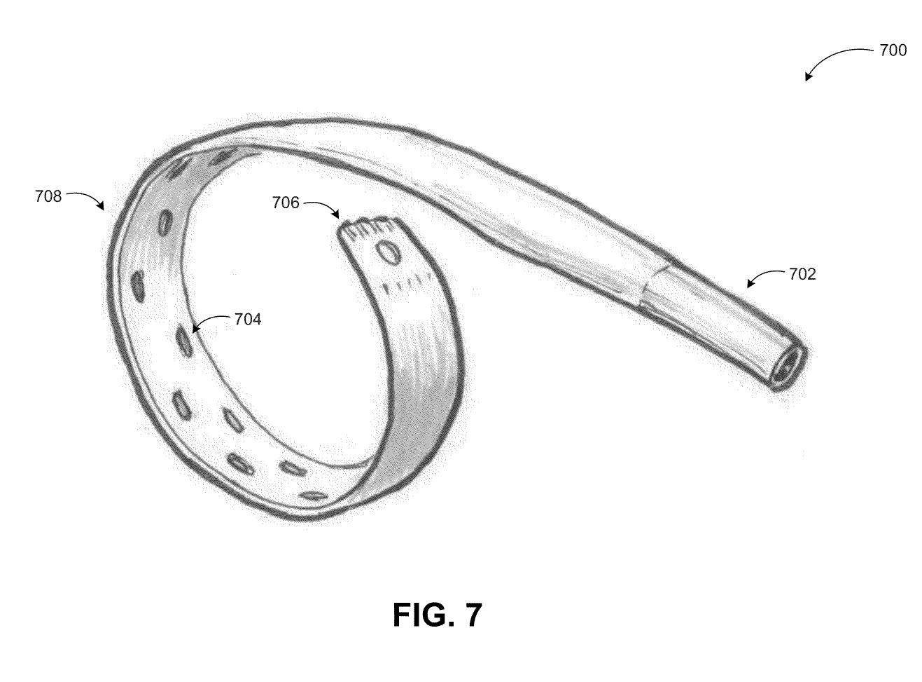

FIG. 7 illustrates another example dual lumen, punctured catheter that may have a substantially flat shape when deployed, arranged in accordance with at least some embodiments described herein.

Diagram 700 shows an alternative configuration of a ribbon-like catheter of FIG. 6 with tubular section 702, flat section 708, and holes 704. Differently from the configuration in FIG. 6, the catheter in diagram 700 may be configured to have a crimped end 706 for fastening the flat section 708 onto itself once the flat section is wrapped around the anastomosis area.

By securing the catheter as a band, it may be possible to restrict the circumferential expansion of the colon due to peristaltic motion that might cause leaks in the first place, effectively preventing the tissue of the newly joined colon from moving and hence promoting healing. This configuration may take some of the strain off the local tissue around the staples or sutures.

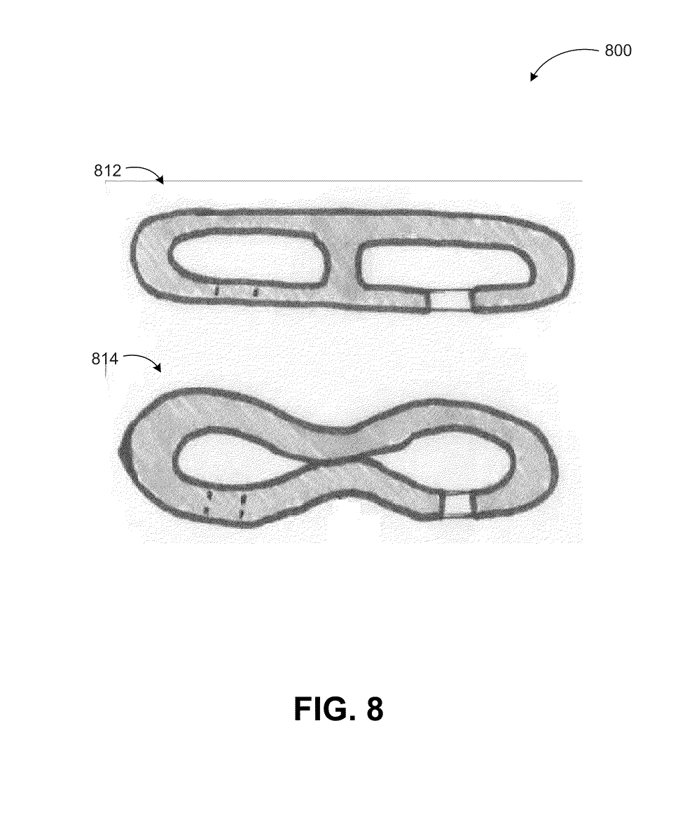

FIG. 8 illustrates two example cross-sectional views of a catheter according to FIG. 6 and FIG. 7, arranged in accordance with at least some embodiments described herein.

Diagram 800 depicts two example cross-section configurations of a ribbon-like catheter as described in FIG. 6 and FIG. 7. As shown in the diagram, one example configuration 812 may include two compartments separated by a middle section, where the cross-section is substantially elliptical. Another example configuration 814 may have a substantially figure eight shaped cross-section, where the middle section may be formed through fusing or welding of the centers of the opposing walls of the catheter effectively forming to isolated compartments, one for injected fluid and another for collected fluid. In either configuration, the holes may be formed on one of the flat walls of the catheter such that they face the anastomosis area (e.g., the colon).

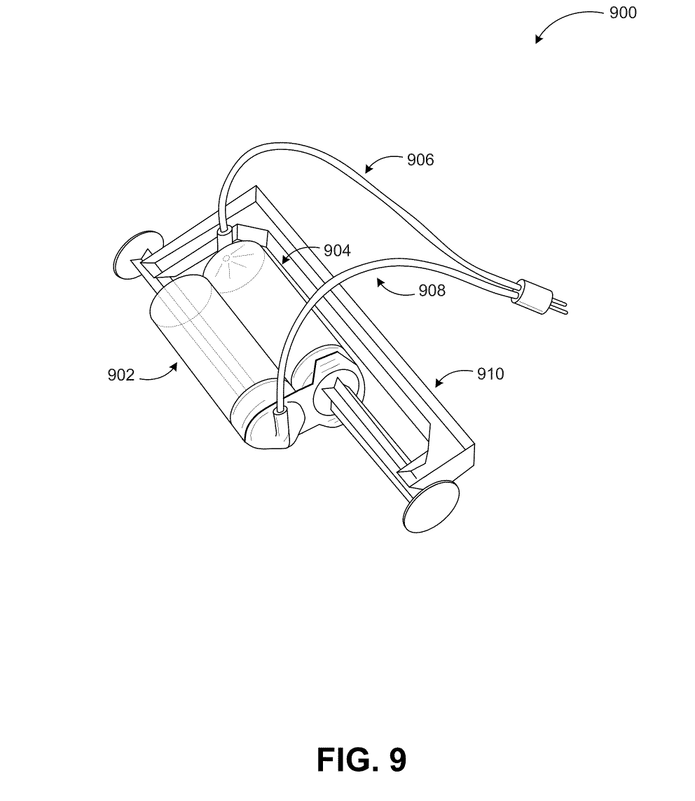

FIG. 9 illustrates an example dual action syringe that may be used a catheter according to FIG. 3 through FIG. 7, arranged in accordance with at least some embodiments described herein.

A number of injection and extraction mechanisms may be employed to inject a neutral fluid to the anastomosis site through a dual action catheter and extract the abdominal fluids in the anastomosis area through the same. Diagram 900 in FIG. 9 show one example syringe configuration, where two distinct syringes 902 and 904 may be affixed together through a bracket 920 or formed as a dual syringe system. When a dual lumen catheter is used, each lumen (906, 908) of the catheter may be coupled to an orifice of the barrel of each syringe.

The bracket 910 may be affixed to the plungers of the syringes 902, 904 such that when the bracket 910 is moved in one direction (relative to the syringes), one of the plungers may push into the barrel of its respective syringe while the other plunger pulls out of the barrel of its respective syringe, thus injecting the fluid in on syringe into the catheter while extracting the fluid in the catheter into the other syringe. In a basic example, the syringes in such a system may be one-time-use syringes and may be removed from the catheter upon use. The extracted fluids may then be emptied into a test device for testing the fluids for the presence of substances indicating anastomotic leakage.

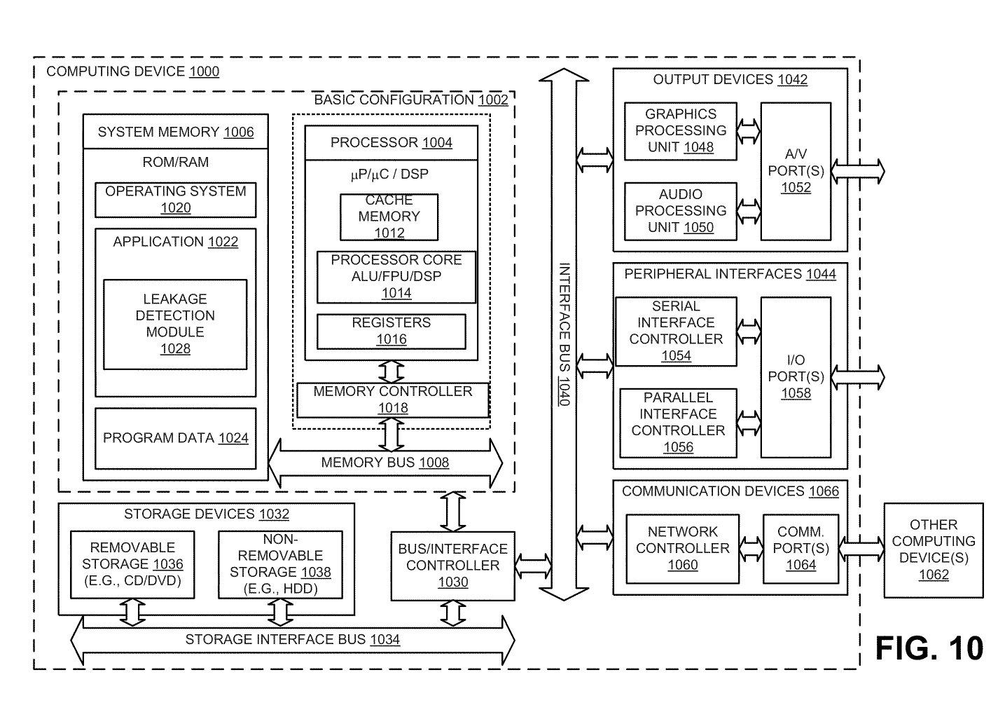

FIG. 10 illustrates a general purpose computing device, which may be used to control a leakage detection system implementing a dual action catheter inside the body, arranged in accordance with at least some embodiments described herein. In a very basic configuration 1002, computing device 1000 typically includes one or more processors 1004 and a system memory 1006. A memory bus 1008 may be used for communicating between processor 1004 and system memory 1006.

Depending on the desired configuration, processor 1004 may be of any type including but not limited to a microprocessor (μP), a microcontroller (μC), a digital signal processor (DSP), or any combination thereof. Processor 1004 may include one more levels of caching, such as a level cache memory 1012, a processor core 1014, and registers 1016. Example processor core 1014 may include an arithmetic logic unit (ALU), a floating point unit (FPU), a digital signal processing core (DSP Core), or any combination thereof. An example memory controller 1018 may also be used with processor 1004, or in some implementations memory controller 1018 may be an internal part of processor 1004.

Depending on the desired configuration, system memory 1006 may be of any type including but not limited to volatile memory (such as RAM), non-volatile memory (such as ROM, flash memory, etc.) or any combination thereof. System memory 1006 may include an operating system 1020, one or more applications 1022, and program data 1024. Application 1022 may include a leakage detection module 1028 that is arranged to detect anastomotic leakage through a dual action catheter and optionally test fluids extracted from the area near the anastomosis. Program data 1024 may include one or more of detection data, and similar data as discussed above. This data may be useful in determining the leakage to the abdominal cavity from an anastomosis. In some embodiments, application 1022 may be arranged to operate with program data 1024 on operating system 1020 such that detection data is analyzed to generate alerts when leakage reaches certain levels as described herein. This described basic configuration 1002 is illustrated in FIG. 10 by those components within the inner dashed line.

Computing device 1000 may have additional features or functionality, and additional interfaces to facilitate communications between basic configuration 1002 and any required devices and interfaces. For example, a bus/interface controller 1030 may be used to facilitate communications between basic configuration 1002 and one or more data storage devices 1032 via a storage interface bus 1034. Data storage devices 1032 may be removable storage devices 1036, non-removable storage devices 1038, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

System memory 1006, removable storage devices 1036 and non-removable storage devices 1038 are examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 1000. Any such computer storage media may be part of computing device 1000.

Computing device 1000 may also include an interface bus 1040 for facilitating communication from various interface devices (e.g., output devices 1042, peripheral interfaces 1044, and communication devices 1046) to basic configuration 1002 via bus/interface controller 1030. Example output devices 1042 include a graphics processing unit 1048 and an audio processing unit 1050, which may be configured to communicate to various external devices such as a display or speakers via one or more A/V ports 1052. Example peripheral interfaces 1044 include a serial interface controller 1054 or a parallel interface controller 1056, which may be configured to communicate with external devices such as input devices (e.g., keyboard, mouse, pen, voice input device, touch input device, etc.) or other peripheral devices (e.g., printer, scanner, etc.) via one or more I/O ports 1058. An example communication device 1046 includes a network controller 1060, which may be arranged to facilitate communications with one or more other computing devices 1062 over a network communication link via one or more communication ports 1064.

The network communication link may be one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

Computing device 1000 may be implemented as a portion of a small-form factor portable (or mobile) electronic device such as a cell phone, a personal data assistant (FDA), a personal computing device, a wireless controller device, an application specific device, or a hybrid device that include any of the above functions. Computing device 1000 may also be implemented as a personal computer including both laptop computer and non-laptop computer configurations. Moreover computing device 1000 may be implemented as a networked system or as part of a general purpose or specialized server.

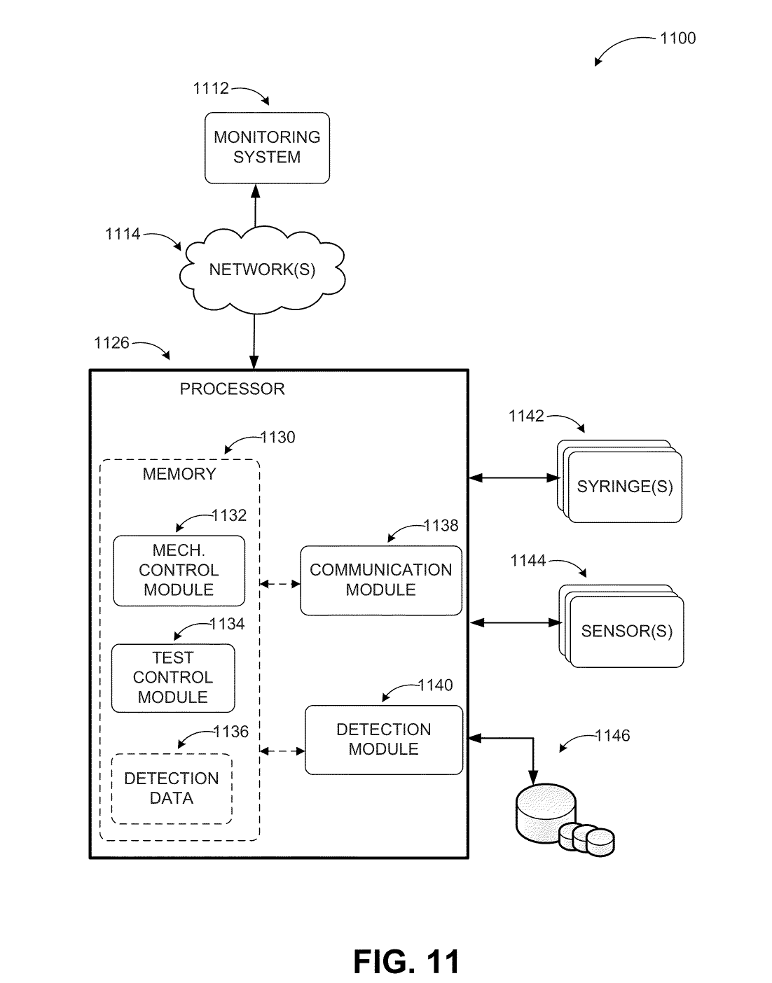

FIG. 11 illustrates a special purpose controller, which may be used to control a dual action syringe and leakage sensors, arranged in accordance with at least some embodiments described herein.

According to some embodiments, an anastomotic leakage detection system may be implemented as a special purpose device with a processor 1126 and one or more components such as syringe(s) 1142 and sensor(s) 1144. Processor 1126 may include special modules such as communication module 1138 and detection module 1140. Detection module 1140 may employ data acquisition including, but not limited to, detection data 1136 from inside the body, which may be stored in memory 1130 or according to other embodiments in remote data stores 1146. The processor 1126 may store in its memory 1130 mechanical control module 1132, test control module 1134, and/or collected detection data 1136. The mechanical control module 1132 may control actions of syringe(s) 1142 causing a neutral fluid to be injected around the area of a surgery site and extraction of abdominal cavity fluids from the same area through a dual action catheter. The test control module 1134 may supervise testing of the extracted fluids to detect substances that may indicate presence of anastomotic leakage. Upon detection of a leakage, the processor 1126 (e.g. through the communication module 1138) may communicate with a monitoring system 1112 via one or more networks 1114 and transmit an alert. Processor 1126 may also receive instructions or data such as calibration data from the monitoring system 1112 in conjunction with performing the tasks described herein.

Example embodiments may also include methods. These methods can be implemented in any number of ways, including the structures described herein. One such way is by machine operations, of devices of the type described in the present disclosure. Another optional way is for one or more of the individual operations of the methods to be performed in conjunction with one or more human operators performing some of the operations while other operations are performed by machines. These human operators need not be collocated with each other, but each can be only with a machine that performs a portion of the program. In other examples, the human interaction can be automated such as by pre-selected criteria that are machine automated.

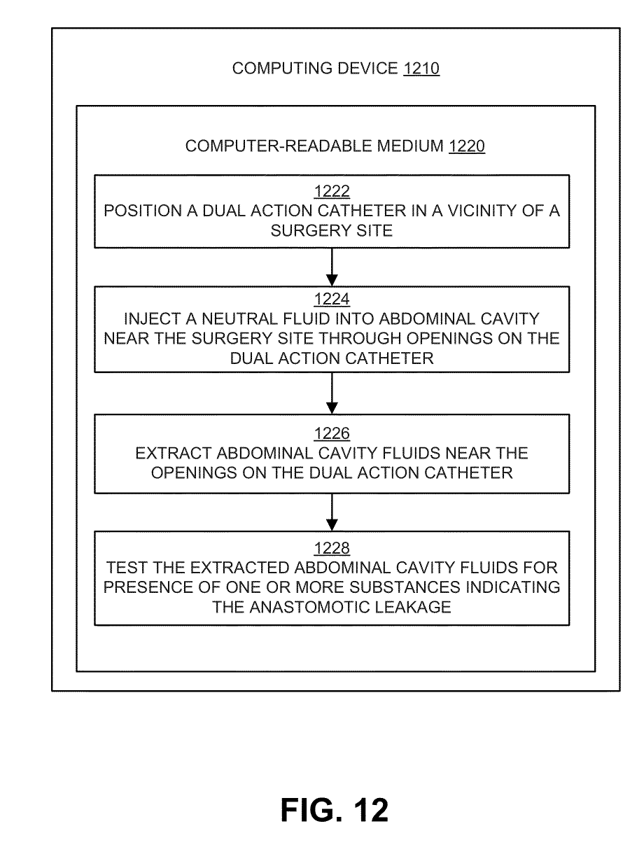

FIG. 12 is a flow diagram illustrating an example method that may be performed by a computing device such as the computing device 700 in FIG. 7 or special purpose processor 1126 in FIG. 11, arranged in accordance with at least some embodiments described herein. Example methods may include one or more operations, functions or actions as illustrated by one or more of blocks 1222, 1224, 1226, and/or 1228. The operations described in blocks 1222 through 1228 may also be stored as computer-executable instructions in a computer-readable medium such as computer-readable medium 1220 of computing device 1210.

A process for detecting the presence of anastomotic leakage in the abdominal cavity through a dual action catheter may begin with block 1222, “POSITION A DUAL ACTION CATHETER IN A VICINITY OF A SURGERY SITE”. At block 1222, a dual action catheter may be placed around the surgery site of an intestine, a portion of a stomach, a colon, an esophagus, or similar organ that been operated on by a surgeon or through a robotic arm at the end of surgery for detection of anastomotic leakage following the surgery.

Block 1222 may be followed by block 1224, “INJECT A NEUTRAL FLUID INTO ABDOMINAL CAVITY NEAR THE SURGERY SITE THROUGH OPENINGS ON THE DUAL ACTION CATHETER.” At block 1224, a neutral fluid such as a saline solution or purified water may be injected through the openings on the dual action catheter (e.g., by action from a syringe system) such that the neutral fluid substantially covers the surgery site outside the organ. The neutral fluid may mix with other abdominal cavity fluids in the immediate area, especially leakage fluids—if there are any.

Block 1224 may be followed by block 1226, “EXTRACT ABDOMINAL CAVITY FLUIDS NEAR THE OPENINGS ON THE DUAL ACTION CATHETER.” At block 1226, the dual action catheter may be used to extract any fluids around the openings through a reverse action of the syringe system, for example. As mentioned above, the fluids near the openings may include the injected neutral fluid and any other fluids that may be in the area such as leaked fluids from the organ.

Block 1226 may be followed by block 1228, “TEST THE EXTRACTED ABDOMINAL CAVITY FLUIDS FOR PRESENCE OF ONE OR MORE SUBSTANCES INDICATING THE ANASTOMOTIC LEAKAGE.” At block 1228, the extracted fluids may be tested for the presence of any substances indicating leakage such as proteins, unprocessed carbohydrates, acidity of the fluids, ingested dyes or radioactive substances, etc.

The blocks included in the above described process are for illustration purposes. Anastomotic leakage detection through a dual action catheter may be performed by similar processes with fewer or additional blocks. In some examples, the blocks may be performed in a different order. In some other examples, various blocks may be eliminated. In still other examples, various blocks may be divided into additional blocks, or combined together into fewer blocks.

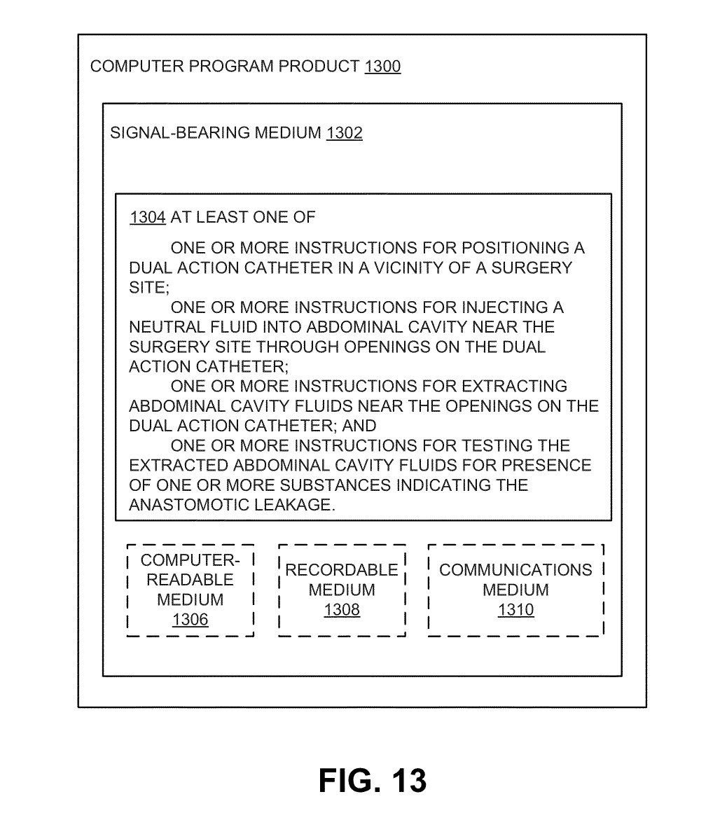

FIG. 13 illustrates a block diagram of an example computer program product, arranged in accordance with at least some embodiments described herein. In some examples, as shown in FIG. 13, computer program product 1300 may include a signal bearing medium 1302 that may also include machine readable instructions 1304 that, when executed by, for example, a processor, may provide the functionality described above with respect to FIG. 10, FIG. 11, and FIG. 12. Thus, for example, referring to processor 1004 or 1126, the leakage detection module 728 or detection module 1140 may undertake one or more of the tasks shown in FIG. 13 in response to instructions 1304 conveyed to processor by medium 1302 to perform actions associated with anastomotic leakage detection through a dual action catheter as described herein. Some of those instructions may include positioning a dual action catheter in a vicinity of a surgery site, injecting a neutral fluid into abdominal cavity near the surgery site through openings on the dual action catheter, extracting abdominal cavity fluids near the openings on the dual action catheter, and testing the extracted abdominal cavity fluids for presence of one or more substances indicating the anastomotic leakage.

In some implementations, signal bearing medium 1302 depicted in FIG. 13 may encompass a computer-readable medium 1306, such as, but not limited to, a hard disk drive, a Compact Disc (CD), a Digital Versatile Disk (DVD), a digital tape, memory, etc. In some implementations, signal bearing medium 1302 may encompass a recordable medium 1308, such as, but not limited to, memory, read/write (R/W) CDs, R/W DVDs, etc. In some implementations, signal bearing medium 1302 may encompass a communications medium 1310, such as, but not limited to, a digital and/or an analog communication medium (e.g., a fiber optic cable, a waveguide, a wired communications link, a wireless communication link, etc.). Thus, for example, program product 1300 may be conveyed to one or more modules of the processor 1004 by an RF signal bearing medium 1302, where the signal bearing medium 1302 is conveyed by a wireless communications medium 1310 (e.g., a wireless communications medium conforming with the IEEE 802.11 standard).

According to some embodiments, the present disclosure describes a method for detecting presence of anastomotic leakage inside a body. An example method may include positioning a dual action catheter in a vicinity of a surgery site, causing a neutral fluid to be injected into abdominal cavity near the surgery site through openings on the dual action catheter, causing abdominal cavity fluids near the openings on the dual action catheter to be extracted, and testing the extracted abdominal cavity fluids for presence of one or more substances indicating the anastomotic leakage.

The method may further include causing the neutral fluid to be injected and the abdominal cavity fluids extracted simultaneously by employing a dual action syringe coupled to the catheter, where the catheter is a dual lumen catheter with one lumen for injecting the neutral fluid and another lumen for extracting the abdominal cavity fluids. The method may also include causing the neutral fluid to be injected and the abdominal cavity fluids extracted sequentially by injecting the neutral fluid and subsequently extracting the abdominal cavity fluids.

The catheter may include a smooth-surface, flexible tube, and positioning the catheter in the vicinity of a surgery site may include forming one or more loops around the surgery site with a portion of the catheter including the openings. Alternatively, the catheter may include two or more branched end-portions including the openings, and positioning the catheter in the vicinity of a surgery site may include affixing the branched end-portions substantially parallel to each other at the surgery site. The catheter may further include a smooth-surface, flexible, meandering tube, and positioning the catheter in the vicinity of a surgery site may include affixing the meandering tube portion of the catheter with the openings at the surgery site.

The catheter may include a substantially tubular first section and a substantially flat second section, and positioning the catheter in the vicinity of a surgery site may include wrapping the substantially flat second section around the surgery site. The substantially flat second section may include the openings along one of its flat surfaces facing the surgery site. The method may also include fastening the substantially flat second section of the catheter through a crimped end of the substantially flat second section following the wrapping, or fastening the substantially flat second section of the catheter through one or more adhesive bands along flat surfaces of the substantially flat second section following the wrapping. The adhesive tape may include a single continuous tape or a plurality of discrete wings.

The method may also include testing the extracted abdominal cavity fluids for presence of one or more of proteins, carbohydrates, selected bacteria, and an increase in pH level. The method may further include testing the extracted abdominal cavity fluids for the presence of one or more of an ingested dye, an ingested radioactive tracer, and ingested activated charcoal particles. The neutral fluid may include a saline solution or purified water. The method may also include affixing the catheter near the vicinity of the surgery site through sutures or a staple system during surgery.

The catheter may include a memory polymer and affixing the catheter near the vicinity of the surgery site may include forming one or more loops around the surgery site, where upon completion of leakage monitoring, the catheter may be removed through a gentle rip-cord action. The catheter may be affixed to an esophagus, a stomach, a small intestine, or a large intestine. The openings may be manifold holes staggered relative to each side of two lumen paths of the catheter such that a longest passage of the neutral fluid is created in the abdominal cavity. An end of the catheter residing in the abdominal cavity may be sealed. The catheter may also include two lumens and a cross-section of the lumens may include a circular shape, an elliptic shape, and a semi-circular shape. A size, a number, and/or a distribution of the openings may be determined based on one or more of a surgery site, a surgery type, and a length of the catheter.

The method may also include performing a calibration to determine a baseline amount of detected leakage inside the abdominal cavity, repeating the injection of the neutral fluid and the extraction of the abdominal cavity fluids at a predefined interval, applying statistical analysis to the collected leakage detection information for determining an amount of fluid leaked from an anastomosis into the abdominal cavity, or injecting one or more of a therapeutic medication and a sterilizing agent near the surgery site through the catheter.

According to other embodiments, the present disclosure also describes a system for detecting presence of anastomotic leakage inside a body. An example system may include a dual action catheter positioned in a vicinity of a surgery site in an abdominal cavity and a dual action syringe system. The dual action catheter may release a neutral fluid into the abdominal cavity near the surgery site through openings on the dual action catheter and collect abdominal cavity fluids near the openings on the dual action catheter. The dual action syringe system may inject the neutral fluid into the dual action catheter and extract the abdominal cavity fluids from the dual action catheter such that the extracted abdominal cavity fluids are tested for presence of one or more substances indicating the anastomotic leakage.

The system may also include a controller configured to manage the dual action syringe system for automated injection of the neutral fluid and extraction of the abdominal cavity fluids. The controller may also be configured to perform a calibration to determine a baseline amount of detected leakage inside the abdominal cavity, repeat the injection of the neutral fluid and the extraction of the abdominal cavity fluids at a predefined interval, or apply statistical analysis to the collected leakage detection information to determine an amount of fluid caked from an anastomosis into the abdominal cavity.

The catheter may be a dual lumen catheter with one lumen for injecting the neutral fluid and another lumen for extracting the abdominal cavity fluids simultaneously. Alternatively, the catheter may be a single lumen catheter for sequential injection of the neutral fluid and extraction of the abdominal cavity fluids. The catheter may also be a smooth-surface, flexible tube, and may be positioned in the vicinity of a surgery site forming one or more loops around the surgery site with a portion of the catheter including the openings. The catheter may further include two or more branched end-portions including the openings, and may be positioned in the vicinity of the surgery site by affixing the branched end-portions substantially parallel to each other at the surgery site. The catheter may further include a smooth-surface, flexible, meandering tube, and may be positioned in the vicinity of the surgery site by affixing the meandering tube portion of the catheter with the openings at the surgery site.

The catheter may include a substantially tubular first section and a substantially flat second section, and may be positioned in the vicinity of a surgery site by wrapping the substantially flat second section around the surgery site. The substantially flat second section may include the openings along one of its flat surfaces facing the surgery site. The substantially flat second section of the catheter may be fastened through a crimped end of the substantially flat second section following the wrapping, or it may be fastened through one or more adhesive bands along flat surfaces of the substantially flat second section following the wrapping. The adhesive tape may include a single continuous tape or a plurality of discrete wings.

The system may further include a test device configured to test the extracted abdominal cavity fluids for presence of one or more of proteins, carbohydrates, selected bacteria, and changes in pH level. The test device may test the extracted abdominal cavity fluids for presence of one or more of an ingested dye, an ingested radioactive tracer, and ingested activated charcoal particles. The neutral fluid may include one of a saline solution and purified water. The catheter may be affixed near the vicinity of the surgery site through sutures, clips or a staple system during surgery.

The catheter may include a memory polymer and may be affixed near the vicinity of the surgery site forming one or more loops around the surgery site. The catheter may be removed through a gentle rip-cord action upon completion of leakage monitoring. The catheter may be affixed to an esophagus, a stomach, a small intestine, or a large intestine. The openings may be manifold holes staggered relative to each side of two lumen paths of the catheter such that a longest passage of the neutral fluid is created in the abdominal cavity. An end of the catheter residing in the abdominal cavity may be sealed. The catheter may include two lumens and a cross-section of the lumens may include one of: a circular shape, an elliptic shape, and a semi-circular shape. A size, a number, and/or a distribution of the openings may be determined based on one or more of a surgery site, a surgery type, and a length of the catheter. The dual action syringe system may further be configured to inject one or more of a therapeutic medication and a sterilizing agent near the surgery site through the catheter.

According to further embodiments, the present disclosure describes a dual action catheter for detecting presence of anastomotic leakage inside a body. The dual action catheter may include a first portion with at least one lumen, where the first portion is arranged to couple to a dual action syringe system outside a body for receiving a neutral fluid to be injected into an abdominal cavity in a vicinity of a surgery site and providing abdominal cavity fluids extracted from the vicinity of the surgery site. The dual action catheter may also include a second portion with at least one lumen, where the second portion is arranged to couple to the first portion and to release the neutral fluid into the abdominal cavity in the vicinity of the surgery site and collect abdominal cavity fluids through openings on the second portion such that the extracted abdominal cavity fluids are tested for presence of one or more substances indicating the anastomotic leakage.

The first portion may include a first lumen for injecting the neutral fluid and a second lumen for extracting the abdominal cavity fluids simultaneously. Alternatively, the first portion may include a single lumen for sequential injection of the neutral fluid and extraction of the abdominal cavity fluids. The second portion may include a smooth-surface, flexible tube, and may be positioned in the vicinity of a surgery site forming one or more loops around the surgery site. The second portion may also include two or more branched end-portions including the openings, and may be positioned in the vicinity of the surgery site by affixing the branched end-portions substantially parallel to each other at the surgery site. Alternatively, the second portion may include a smooth-surface, flexible, meandering tube, and may be positioned in the vicinity of the surgery site by affixing the meandering tube with the openings at the surgery site.

The second portion may be substantially tubular and the second portion may be substantially flat. The openings may be along one two flat surfaces of the substantially flat second portion facing the surgery site. The substantially flat second portion may be fastened through a crimped end of the substantially flat second portion, or it may be fastened through one or more adhesive bands along flat surfaces of the substantially flat second portion. The adhesive tape may include a single continuous tape or a plurality of discrete wings.

The second portion may be affixed near the vicinity of the surgery site through sutures or a staple system during surgery. The second portion may include a memory polymer and may be affixed near the vicinity of the surgery site forming one or more loops around the surgery site. The openings may be manifold holes staggered relative to each side of two lumen paths of the catheter such that a longest passage of the neutral fluid is created in the abdominal cavity. An end of the second portion residing in the abdominal cavity may be sealed. The catheter may include two lumens and a cross-section of the lumens may include a circular shape, an elliptic shape, or a semi-circular shape. A size, a number, and/or a distribution of the openings may be determined based on one or more of a surgery site, a surgery type, and a length of the catheter. The catheter may also be used for injecting one or more of a therapeutic medication and a sterilizing agent near the surgery site through the catheter.

According to yet other embodiments, the present disclosure describes a computer readable memory device with instructions stored thereon for controlling a dual action catheter based system to detect presence of anastomotic leakage inside a body. The instructions may include causing a neutral fluid to be injected into abdominal cavity near a surgery site through openings on a dual action catheter, where the catheter is positioned in a vicinity of the surgery site; causing abdominal cavity fluids near the openings on the dual action catheter to be extracted; and testing the extracted abdominal cavity fluids for presence of one or more substances indicating the anastomotic leakage.

The instructions may further include causing the neutral fluid to be injected and the abdominal cavity fluids extracted simultaneously by employing a dual action syringe coupled to the catheter, where the catheter is a dual lumen catheter with one lumen for injecting the neutral fluid and another lumen for extracting the abdominal cavity fluids. The instructions may also include causing the neutral fluid to be injected and the abdominal cavity fluids extracted sequentially by injecting the neutral fluid and subsequently extracting the abdominal cavity fluids.

The catheter may include a smooth-surface, flexible tube, and positioning the catheter in the vicinity of a surgery site may include forming one or more loops around the surgery site with a portion of the catheter including the openings. Alternatively, the catheter may include two or more branched end-portions including the openings, and positioning the catheter in the vicinity of a surgery site may include affixing the branched end-portions substantially parallel to each other at the surgery site. The catheter may further include a smooth-surface, flexible, meandering tube, and positioning the catheter in the vicinity of a surgery site may include affixing the meandering tube portion of the catheter with the openings at the surgery site.

The instructions may also include testing the extracted abdominal cavity fluids for presence of one or more of proteins, carbohydrates, selected bacteria, and an increase in pH level. The instructions may further include testing the extracted abdominal cavity fluids for the presence of one or more of an ingested dye, an ingested radioactive tracer, and ingested activated charcoal particles. The neutral fluid may include a saline solution or purified water. The instructions may also include affixing the catheter near the vicinity of the surgery site through sutures or a staple system during surgery.

The catheter may include a memory polymer and/or re-absorbable polymer) and affixing the catheter near the vicinity of the surgery site may include forming one or more loops around the surgery site, where upon completion of leakage monitoring, the catheter may be removed through a gentle rip-cord action. A numbing agent (anesthetic) may be injected via the catheter before removal to minimize any discomfort. The catheter may be affixed to an esophagus, a stomach, a small intestine, or a large intestine. The openings may be manifold holes staggered relative to each side of two lumen paths of the catheter such that a longest passage of the neutral fluid is created in the abdominal cavity. An end of the catheter residing in the abdominal cavity may be sealed. The catheter may also include two lumens and a cross-section of the lumens may include a circular shape, an elliptic shape, and a semi-circular shape. A size, a number, and/or a distribution of the openings may be determined based on one or more of a surgery site, a surgery type, and a length of the catheter.

The instructions may also include performing a calibration to determine a baseline amount of detected leakage inside the abdominal cavity, repeating the injection of the neutral fluid and the extraction of the abdominal cavity fluids at a predefined interval, applying statistical analysis to the collected leakage detection information for determining an amount of fluid leaked from an anastomosis into the abdominal cavity, or injecting one or more of a therapeutic medication and a sterilizing agent near the surgery site through the catheter.

According to yet further embodiments, the present disclosure describes a method for detecting presence of anastomotic leakage inside a body. The method may include positioning a dual action catheter in a vicinity of a surgery site, causing a neutral fluid to be injected into abdominal cavity near the surgery site through openings on the dual action catheter, and causing abdominal cavity fluids near the openings on the dual action catheter to be extracted. The method may further include testing the extracted abdominal cavity fluids for presence of one or more substances indicating the anastomotic leakage. The catheter may include a smooth-surface, flexible tube, and positioning the catheter in the vicinity of a surgery site may include forming one or more loops around the surgery site with a portion of the catheter including the openings. The catheter may also include two or more branched end-portions including the openings, and positioning the catheter in the vicinity of a surgery site may also include affixing the branched end-portions substantially parallel to each other at the surgery site. Alternatively. The catheter may further include a smooth-surface, flexible, meandering tube, and positioning the catheter in the vicinity of a surgery site may further include affixing the meandering tube portion of the catheter with the openings at the surgery site.

There is little distinction left between hardware and software implementations of aspects of systems; the use of hardware or software is generally (but not always, in that in certain contexts the choice between hardware and software may become significant) a design choice representing cost vs. efficiency tradeoffs. There are various vehicles by which processes and/or systems and/or other technologies described herein may be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples may be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, may be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Versatile Disk (DVD), a digital tape, a computer memory, a solid state drive, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein may be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity of gantry systems; control motors for moving and/or adjusting components and/or quantities).

A typical data processing system may be implemented utilizing any suitable commercially available components, such as those typically found in data computing/communication and/or network computing/communication systems. The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermediate components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically connectable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for detecting presence of anastomotic leakage inside a body, the method comprising:
positioning a dual action catheter in a vicinity of a surgery site;
causing a neutral fluid to be injected into abdominal cavity near the surgery site through openings on the dual action catheter;
causing abdominal cavity fluids near the openings on the dual action catheter to be extracted, wherein the neutral fluid is injected and the abdominal cavity fluids are extracted simultaneously by employing a dual action syringe coupled to the dual action catheter; and
testing the extracted abdominal cavity fluids for presence of one or more substances indicating the anastomotic leakage.

2.-14. (canceled)

15. The method of claim 1, further comprising:
affixing the catheter near the vicinity of the surgery site through sutures or a staple system during surgery.

16. The method of claim 15, wherein the catheter comprises a memory polymer and affixing the catheter near the vicinity of the surgery site comprises forming one or more loops around the surgery site.

17. The method of claim 16, further comprising:
upon completion of leakage monitoring, removing the catheter through a gentle rip-cord action.

18.-22. (canceled)

23. The method of claim 1, further comprising:
performing a calibration to determine a baseline amount of detected leakage inside the abdominal cavity.

24. The method of claim 1, further comprising:
repeating the injection of the neutral fluid and the extraction of the abdominal cavity fluids at a predefined interval.

25. The method of claim 1, further comprising:
applying statistical analysis to collected leakage detection information for determining an amount of fluid leaked from an anastomosis into the abdominal cavity.

26. (canceled)

27. A system for detecting presence of anastomotic leakage inside a body, the system comprising:
a dual action catheter positioned in a vicinity of a surgery site in an abdominal cavity, the dual action catheter configured to release a neutral fluid into the abdominal cavity near the surgery site through openings on the dual action catheter and collect abdominal cavity fluids near the openings on the dual action catheter; and
a dual action syringe system configured to inject the neutral fluid into the dual action catheter and extract the abdominal cavity fluids from the dual action catheter simultaneously such that the extracted abdominal cavity fluids are tested for presence of one or more substances indicating the anastomotic leakage.

28. The system of claim 27, further comprising:
a controller configured to manage the dual action syringe system for automated injection of the neutral fluid and extraction of the abdominal cavity fluids.

29.-41. (canceled)

42. The system of claim 27, further comprising:
a test device configured to test the extracted abdominal cavity fluids for presence of one or more of proteins, carbohydrates, selected bacteria, an increase in pH level, an ingested dye, an ingested radioactive tracer, and ingested activated charcoal particles.

43. (canceled)

44. The system of claim 27, wherein the neutral fluid includes one of a saline solution and purified water.

45.-48. (canceled)

49. The system of claim 27, wherein the openings are manifold holes staggered relative to each side of two lumen paths of the catheter such that a longest passage of the neutral fluid is created in the abdominal cavity, and at least one of a size, number, and distribution of the openings are determined based on one or more of a surgery site, a surgery type, and a length of the catheter.

50.-53. (canceled)

54. A dual action catheter for detecting presence of anastomotic leakage inside a body, the catheter comprising:
a first portion comprising at least one lumen, the first portion configured to couple to a dual action syringe system outside a body for receiving a neutral fluid to be injected into an abdominal cavity in a vicinity of a surgery site and providing abdominal cavity fluids extracted from the vicinity of the surgery site, wherein the neutral fluid is injected and the abdominal cavity fluids are extracted simultaneously; and
a second portion comprising at least one lumen, the second portion configured to couple to the first portion and to release the neutral fluid into the abdominal cavity in the vicinity of the surgery site and collect abdominal cavity fluids through openings on the second portion such that the extracted abdominal cavity fluids are tested for presence of one or more substances indicating the anastomotic leakage.

55. The dual action catheter of claim 54, wherein the first portion includes a first lumen for injecting the neutral fluid and a second lumen for extracting the abdominal cavity fluids simultaneously.

56. The dual action catheter of claim 54, wherein the first portion includes a single lumen for sequential injection of the neutral fluid and extraction of the abdominal cavity fluids.

57. The dual action catheter of claim 54, wherein the second portion comprises a smooth-surface, flexible tube, and is positioned in the vicinity of a surgery site forming one or more loops around the surgery site.

58. The dual action catheter of claim 54, wherein the second portion comprises two or more branched end-portions including the openings, and is positioned in the vicinity of the surgery site by affixing the branched end-portions substantially parallel to each other at the surgery site.

59. The dual action catheter of claim 54, wherein the second portion comprises a smooth-surface, flexible, meandering tube, and is positioned in the vicinity of the surgery site by affixing the meandering tube with the openings at the surgery site.

60. The dual action catheter of claim 54, wherein the second portion is substantially tubular and substantially flat such that the openings are along at least one flat surface of the second portion facing the surgery site.

61. (canceled)

62. The dual action catheter of claim 60, wherein the substantially flat second portion is one of: fastened through a crimped end of the substantially flat second portion and fastened through one or more adhesive bands along flat surfaces of the substantially flat second portion.

63.-73. (canceled)
